(19) United States
(12) Patent Application Publication
(10) Pub. No.: US 2026/0158100 A1
(43) Pub. Date: Jun. 11, 2026

(54) ENZYME FOOD FOR MAINTAINING BODY BALANCE AND PREVENTING METABOLIC SYNDROME THROUGH ENZYMES

(71) Applicant: Apharm Health Co., Ltd., Daegu (KR)

(72) Inventor: Young HUH, Daegu (KR)

(21) Appl. No.: 19/202,536

(22) Filed: May 8, 2025

(30) Foreign Application Priority Data
Dec. 5, 2024 (KR) 10-2024-0179066

(51) Int. Cl. A61K 36/899; A61K 9/00; A61K 31/191; A61K 31/702; A61K 31/721; A61K 31/736; A61K 35/747; A61K 36/236; A61K 36/28; A61K 36/68; A61K 36/73; A61K 38/16; A61K 38/43; A61K 45/06

(52) U.S. Cl. CPC A61K 36/899; A61K 9/0053; A61K 31/191; A61K 31/702; A61K 31/721; A61K 31/736; A61K 35/747; A61K 36/236; A61K 36/28; A61K 36/68; A61K 36/73; A61K 38/168; A61K 38/43; A61K 45/06

(57) ABSTRACT

An embodiment relates to an enzyme food that improves dieting effects while also supporting cardiovascular health, anti-aging, and prevention of metabolic syndrome. The enzyme food includes an enzyme preparation including teff enzyme, a powder preparation, dextrin, fructo-oligosaccharides, isolated soy protein, a synthetic flavoring agent, amino acid mixture powder, gluten-degrading lactic acid bacteria, guar gum, vinegar powder, lactic acid bacteria mixture powder, salt, psyllium husk powder, malic acid, chicory extract, and stevia.

### CROSS REFERENCE TO RELATED APPLICATION

The present application claims priority to Korean Patent Application No. 10-2024-0179066, filed Dec. 5, 2024, the entire contents of which are incorporated here for all purposes by this reference.

### BACKGROUND

### Technical Field

The present disclosure relates to an enzyme food, and more particularly, to an enzyme food for maintaining body balance and preventing metabolic syndrome through enzymes.

### Background Art

Unless otherwise indicated herein, the description in this section is not prior art to the claims of the present application and is not admitted to be prior art by inclusion in this section.

Enzyme food refers to a product obtained by culturing edible microorganisms in plant-based ingredients such that the ingredients contain a large amount of enzymes, or a product obtained by extracting an enzyme-containing portion from food, or a product processed by using these as a main ingredient to facilitate ingestion.

Enzyme food has the efficacy of promoting digestion and maintaining intestinal health for modern individuals exposed to diets including carbohydrates such as bread, rice, and noodles, instant foods, delivered meals, or irregular eating patterns, and research and development are increasing due to the demand from these individuals.

Enzyme foods primarily based on enzymes are categorized according to their source materials, into cereal enzyme-containing products, germ enzyme-containing products, fruit and vegetable enzyme-containing products, and other plant enzyme-containing products, and products generally recognized by consumers as enzyme foods often have high-calorie ingredients added to improve taste.

Consumption of such high-calorie enzyme foods leads to obesity, characterized by excessive fat accumulation in the body due to an imbalance between energy intake and expenditure, and obesity contributes to lifestyle-related diseases such as diabetes, hypertension, cardiovascular diseases, hyperlipidemia, and metabolic syndrome.

Therefore, because consumption of high-calorie enzyme foods may lead to obesity, and obesity may in turn lead to lifestyle-related diseases such as diabetes, hypertension, cardiovascular diseases, hyperlipidemia, and metabolic syndrome, there is a need for the development of enzyme foods that are low in calories, aid in weight management, support cardiovascular health, and possess a taste preferred by consumers.

In this regard, Korean Patent Registration No. 10-1866468 discloses enzyme foods and diet foods using an enzyme-concentrated fermented product obtained through microbial grain culture, and methods of manufacturing the same, and Korean Patent Publication No. 10-2325467 discloses a method of manufacturing a fermented enzyme using natural minerals from volcanic seawater, and enzyme food manufactured thereby.

However, existing enzyme foods primarily based on enzymes do not disclose techniques for additionally improving taste and other efficacies beyond the effect of aiding diet management.

### DISCLOSURE OF INVENTION

### Technical Problem

The present disclosure is to provide an enzyme food that improves dieting effects while also supporting cardiovascular health, anti-aging, and prevention of metabolic syndrome.

In addition, the technical problems are not limited to those described above, and it will be apparent that other technical problems may also be derived from the following description.

### Solution to Problem

According to an embodiment disclosed herein, an enzyme food includes an enzyme preparation including teff enzyme, a powder preparation, dextrin, fructo-oligosaccharides, isolated soy protein, a synthetic flavoring agent, amino acid mixture powder, gluten-degrading lactic acid bacteria, guar gum, vinegar powder, lactic acid bacteria mixture powder, salt, psyllium husk powder, malic acid, chicory extract, and stevia.

In addition, the enzyme food may include 29 to 40 parts by weight of the enzyme preparation, 26 to 37 parts by weight of the powder preparation, 5 to 9 parts by weight of dextrin, 2 to 5 parts by weight of fructo-oligosaccharides, 1 to 4 parts by weight of isolated soy protein, 0.375 to 2 parts by weight of the synthetic flavoring agent, 0.2 to 2 parts by weight of the amino acid mixture powder, 0.1 to 2 parts by weight of the gluten-degrading lactic acid bacteria, 0.05 to 2 parts by weight of guar gum, 0.05 to 2 parts by weight of the vinegar powder, 0.05 to 2 parts by weight of the lactic acid bacteria mixture powder, 0.05 to 2 parts by weight of salt, 0.05 to 2 parts by weight of the psyllium husk powder, 0.005 to 2 parts by weight of malic acid, 0.005 to 2 parts by weight of the chicory extract, and 0.005 to 2 parts by weight of stevia.

In addition, the enzyme food may further include 8 to 13 parts by weight of apple powder. In addition, the enzyme food may further include 0.15 to 2 parts by weight of licorice concentrate powder.

In addition, the enzyme preparation may include, per 100 parts by weight of the enzyme preparation, 50 parts by weight of teff enzyme, 20 parts by weight of vinegar powder, and 30 parts by weight of gluten-degrading lactic acid bacteria.

In addition, the powder preparation may be indigestible maltodextrin, Bacillus subtilis, Aspergillus oryzae, cereal mixture powder, or germinated brown rice.

In addition, the stevia may be enzyme-treated stevia manufactured by adding glycosyl enzyme to stevia extract and fermenting a resulting mixture.

In addition, the lactic acid bacteria mixture powder may include Lactobacillus plantarum, Lactobacillus rhamnosus, Lactobacillus casei, Lactobacillus acidophilus, Lactobacillus paracasei, Lactobacillus salivarius, and Lactobacillus gasseri.

In addition, the enzyme food may include 38.8 parts by weight of the enzyme preparation, 35.22 parts by weight of the powder preparation, 11.4 parts by weight of the apple powder, 7.2 parts by weight of dextrin, 3.1 parts by weight of fructo-oligosaccharides, 2.1 parts by weight of isolated soy protein, 0.75 parts by weight of the synthetic flavoring agent, 0.4 parts by weight of the amino acid mixture powder, 0.3 parts by weight of the licorice concentrate powder, 0.2 parts by weight of the gluten-degrading lactic acid bacteria, 0.1 parts by weight of guar gum, 0.1 parts by weight of the vinegar powder; 0.1 parts by weight of the lactic acid bacteria mixture powder, 0.1 parts by weight of salt, 0.1 parts by weight of the psyllium husk powder, 0.01 parts by weight of malic acid, 0.01 parts by weight of the chicory extract, and 0.01 parts by weight of the enzyme-treated stevia.

### Advantageous Effects of Invention

According to an embodiment disclosed herein, the apple powder, fructo-oligosaccharides, psyllium husk powder, gluten-degrading lactic acid bacteria, and chicory extract, which are included in the enzyme food, are commonly rich in dietary fiber and prebiotics, and thus offer the advantages of being effective for weight loss, promoting beneficial intestinal bacteria growth, improving intestinal health, and aiding diet improvement.

The apple powder, dextrin, fructo-oligosaccharides, isolated soy protein, licorice concentrate powder, guar gum, vinegar powder, salt, psyllium husk powder, malic acid, chicory extract, and enzyme-treated stevia, which are contained in the enzyme food, are commonly specialized in normalizing cholesterol levels. Through a synergistic effect of cholesterol level normalization and diet improvement effects, they offer the advantage of preventing heart diseases including arteriosclerosis, angina, and myocardial infarction, cerebrovascular diseases including stroke and hypertension, and metabolic syndrome.

The gluten-degrading lactic acid bacteria, fructo-oligosaccharides, and apple powder, which are included in the enzyme food, increase beneficial intestinal bacteria and help activate immune cells. As intestinal health improves, the immune system strengthens, increasing resistance to external pathogens.

The psyllium husk, chicory, and guar gum, which are included in the enzyme food, help prevent overeating by slowing the digestion rate and maintaining satiety, and also contribute to blood sugar control by preventing rapid blood sugar spikes.

The apple powder, licorice concentrate powder, and vinegar powder, which are contained in the enzyme food, include antioxidant components, which help remove oxygen free radicals from the body, and polyphenol components contained in apples provide a strong antioxidant effect, contributing to reduced cell damage.

The isolated soy protein and amino acids, which are contained in the enzyme food, supplement the body's protein and aid recovery from muscle damage. They are effective for maintaining physical energy and reducing fatigue.

The apple powder, guar gum, psyllium husk powder, and vinegar powder, which are contained in the enzyme food, help lower blood cholesterol levels, and pectin contained in the apple powder, and the guar gum play roles in lowering cholesterol, while the vinegar powder contributes to blood pressure management.

The apple powder, fructo-oligosaccharides, isolated soy protein, amino acid mixture powder, licorice concentrate powder, gluten-degrading lactic acid bacteria, guar gum, vinegar powder, lactic acid bacteria mixture powder, psyllium husk powder, malic acid, chicory extract, and enzyme-treated stevia, which are contained in the enzyme food, commonly possess anti-aging efficacy. Therefore, the enzyme food offers the advantage of being effective for anti-aging in addition to supporting intestinal and cardiovascular health.

In addition, the effects of the present disclosure are naturally exhibited by the configurations to be described below, regardless of whether the inventor recognizes them, and thus, the above-described effects are merely a few effects based on the following descriptions, and it should not be recognized that the inventor has described all the effects that have been figured out or are present. In addition, the effects of the present disclosure should be further determined by the overall description of the specification. Even when an effect is not described in an explicit sentence but it can be recognized through the present specification by a person skilled in the art to which the present disclosure pertains, it should be regarded as the effect described in the present specification.

### BRIEF DESCRIPTION OF DRAWINGS

FIG. 1 shows ingredients included in an enzyme food according to an embodiment of the present disclosure.

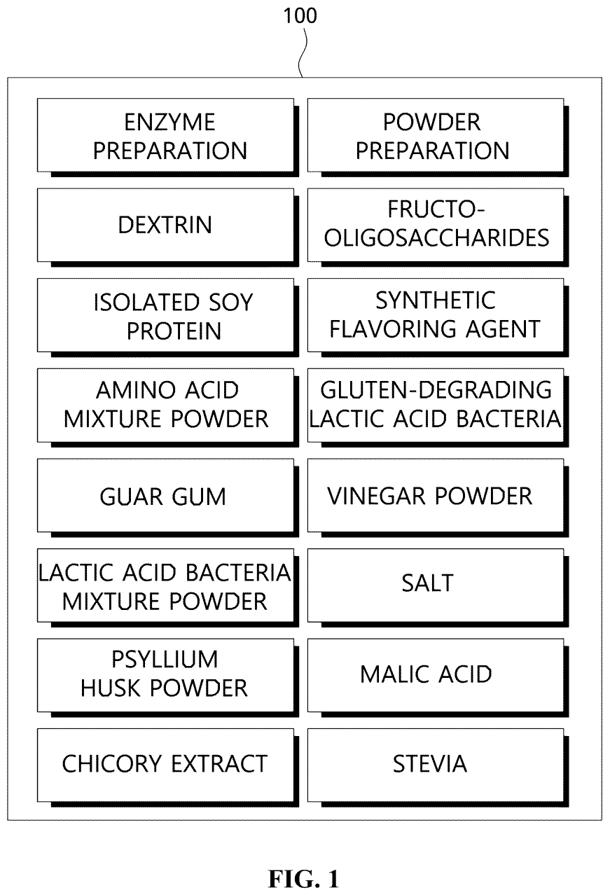

### MODE FOR THE INVENTION

Hereinafter, a configuration, operation, and effects of an enzyme food according to a preferred embodiment will be described with reference to the accompanying drawings. It should be noted that in the accompanying drawings, components may be omitted or schematically illustrated for convenience and clarity, and the size of each component does not necessarily reflect its actual scale. Furthermore, like reference numerals refer to like components throughout the specification, and reference numerals for identical components may be omitted in individual drawings.

An enzyme food 100 according to an embodiment of the present disclosure includes an enzyme preparation, a powder preparation, apple powder, dextrin, fructo-oligosaccharides, isolated soy protein, a synthetic flavoring agent, amino acid mixture powder, licorice concentrate powder, gluten-degrading lactic acid bacteria, guar gum, vinegar powder, lactic acid bacteria mixture powder, salt, psyllium husk powder, malic acid, chicory extract, and enzyme-treated stevia.

TABLE 1

| | Minimum | Maximum | Example |
|---|---|---|---|
| | (parts | (parts | (parts |
| Ingredient | by weight) | by weight) | by weight) |
| Enzyme preparation | 29 | 40 | 38.8 |
| Powder preparation | 26 | 37 | 35.22 |
| Apple powder | 8 | 13 | 11.4 |
| Dextrin | 5 | 9 | 7.2 |
| Fructo- | 2 | 5 | 3.1 |
| oligosaccharides | | | |
| Isolated soy protein | 1 | 4 | 2.1 |
| Synthetic flavoring | 0.375 | 2 | 0.75 |
| agent (apple flavor) | | | |
| Amino acid mixture | 0.2 | 2 | 0.4 |
| powder | | | |
| Licorice concentrate | 0.15 | 2 | 0.3 |
| powder | | | |
| Gluten-degrading | 0.1 | 2 | 0.2 |
| lactic acid bacteria | | | |
| Guar gum | 0.05 | 2 | 0.1 |
| Vinegar powder | 0.05 | 2 | 0.1 |
| Lactic acid bacteria | 0.05 | 2 | 0.1 |
| mixture powder | | | |
| Salt | 0.05 | 2 | 0.1 |
| Psyllium husk | 0.05 | 2 | 0.1 |
| powder | | | |
| Malic acid | 0.005 | 2 | 0.01 |
| Chicory extract | 0.005 | 2 | 0.01 |
| Enzyme-treated | 0.005 | 2 | 0.01 |
| stevia | | | |
| Total | 72.09 | 132 | 100 |

In detail, referring to Table 1 above, the enzyme food 100 includes 38.8 parts by weight of an enzyme preparation, 35.22 parts by weight of a powder preparation, 11.4 parts by weight of apple powder, 7.2 parts by weight of dextrin, 3.1 parts by weight of fructo-oligosaccharides, 2.1 parts by weight of isolated soy protein, 0.75 parts by weight of a synthetic flavoring agent, 0.4 parts by weight of amino acid mixture powder, 0.3 parts by weight of licorice concentrate powder, 0.2 parts by weight of gluten-degrading lactic acid bacteria, 0.1 parts by weight of guar gum, 0.1 parts by weight of vinegar powder, 0.1 parts by weight of lactic acid bacteria mixture powder, 0.1 parts by weight of salt, 0.1 parts by weight of psyllium husk powder, 0.01 parts by weight of malic acid, 0.01 parts by weight of chicory extract, and 0.01 parts by weight of enzyme-treated stevia.

The enzyme food 100 offers the advantage of improved user palatability due to the inclusion of apple powder, dextrin, fructo-oligosaccharides, a synthetic flavoring agent, licorice concentrate powder, and enzyme-treated stevia.

In addition, the enzyme food 100 has the advantage of improved user palatability because acidity is increased by vinegar powder, lactic acid bacteria mixture powder, and malic acid, while a salty taste is added by salt.

The powder preparation may be digestion-resistant maltodextrin, Bacillus subtilis, Aspergillus oryzae, cereal mixture powder, or germinated brown rice, and may be a combination of digestion-resistant maltodextrin, Bacillus subtilis, Aspergillus oryzae, cereal mixture powder, and germinated brown rice.

The enzyme preparation included in the enzyme food 100 may include, per 100 parts by weight of the enzyme preparation, 50 parts by weight of teff enzyme, 20 parts by weight of vinegar powder, and 30 parts by weight of gluten-degrading lactic acid bacteria, or may consist entirely of 100 parts by weight of teff enzyme.

Teff, an annual grass of the Poaceae family used to manufacture teff enzyme, originates from the Horn of Africa, is cultivated as a staple grain in Ethiopia and Eritrea, and contains various nutrients.

Teff enzyme is manufactured by concentrating effective nutrients from teff via a fermentation process and during the fermentation process, specific beneficial microorganisms are used to further activate teff's nutrients, thereby enhancing beneficial compounds such as digestive enzymes.

The teff enzyme is manufactured via a preparation operation involving washing and processing teff to be suitable for fermentation, a fermentation operation involving fermenting the teff at a constant temperature and humidity while adding fermentation microorganisms, such as lactic acid bacteria or yeast, such that teff components are decomposed and transformed, and an enzyme extraction operation involving filtration and concentration processes after the fermentation is complete, allowing extraction of enzymes and active ingredients for product processing.

Teff is rich in amino acids and includes proteins such as lysine, which aids in muscle generation and tissue recovery, and also contains insoluble dietary fiber, which improves digestive health, prevents constipation, and promotes the growth of beneficial intestinal bacteria.

Iron and calcium contained in teff help prevent anemia and support bone health, and iron aids in oxygen transport in the blood, while calcium plays a crucial role in the formation of bones and teeth.

B vitamins contained in teff, such as vitamins B1, B2, and B3, play important roles in energy production and nerve function, vitamin B1 particularly supports nerve and muscle function, and phenolic compounds contained in teff, which are antioxidants, are effective for anti-aging and suppressing inflammation by removing oxygen free radicals and preventing cell damage.

The activation of inherent nutrients of teff via fermentation and enzymatic processes enables the efficacy of the teff enzyme to act more effectively, and the teff enzyme primarily exhibits effects such as promoting amino acid absorption, providing probiotic benefits, and enhancing iron and calcium absorption.

In detail, during the enzymatic process for the teff enzyme, protein-degrading enzymes are activated, facilitating protein digestion and absorption, and enzymatically treated amino acids contained in the teff enzyme are rapidly absorbed by the body, aiding muscle recovery after exercise and contributing to tissue generation and maintenance, and these absorbed amino acids support energy production, helping to alleviate fatigue and enhance vitality.

Dietary fiber contained in the teff enzyme is effective for promoting the growth of beneficial intestinal bacteria and maintaining intestinal health, and soluble fiber contained in the teff enzyme helps manage diabetes by mitigating blood sugar spikes.

Enzymatically treated iron contained in the teff enzyme exhibits increased absorption within the body, making it effective for preventing anemia and forming hemoglobin, and calcium contained in the teff enzyme shows increased absorption, making it effective for preventing osteoporosis and maintaining the strength of bones and teeth.

Vitamin B1 (thiamine) contained in the teff enzyme supports nerve and muscle function and participates in energy production, thereby enhancing stamina and concentration, and vitamin B2 (riboflavin) is effective for maintaining skin and eye health and preventing cell damage through its antioxidant activity.

Vitamin B2 (riboflavin) contained in the teff enzyme prevents cell damage via antioxidant activity and maintains skin and eye health, and vitamin B3 (niacin) contained in the teff enzyme improves blood circulation and regulates blood cholesterol levels, thereby supporting cardiovascular health.

Antioxidant components contained in the teff enzyme, including polyphenols and phenolic compounds, are effective for preventing cell damage, anti-aging, and suppressing inflammation by neutralizing oxygen free radicals, and in particular, they also strengthen immunity by activating the function of immune cells.

The manufacturing of vinegar powder includes a concentration operation to lower the moisture content of vinegar, a drying process in which concentrated vinegar is made into powder via a spray drying or freeze drying method, and a powdering operation of grinding the dried vinegar into powder.

Acetic acid contained in vinegar powder is effective for suppressing post-meal blood sugar surges, improving insulin sensitivity, preventing and managing diabetes, inhibiting fat cell accumulation, reducing body fat, stimulating gastric juice secretion, promoting digestion, and alleviating indigestion.

Organic acids contained in vinegar powder, such as citric acid and malic acid, are effective for recovering from fatigue, alleviating muscle fatigue by removing lactic acid from the body, and maintaining the body's acid-alkaline balance.

Antioxidant components contained in vinegar powder, such as polyphenols, contribute to preventing aging by neutralizing oxygen free radicals and preventing cell damage, and they are also effective for preventing chronic diseases and strengthening immunity by suppressing inflammatory responses.

Amino acids contained in vinegar powder are effective for stabilizing nerves, relieving fatigue, strengthening immune function, enhancing vitality, supporting liver detoxification, and maintaining liver health, and potassium contained in vinegar powder is effective for promoting sodium excretion, regulating blood pressure, supporting cardiovascular health, maintaining electrolyte balance, and regulating body fluid balance.

Gluten-degrading lactic acid bacteria are lactic acid bacteria that degrade gluten, which is an insoluble protein responsible for gluten-sensitive enteropathy, gluten intolerance, gluten allergy, and flour addiction, and these bacteria offer the advantage of promoting the digestion of flour-based foods.

Gluten-degrading lactic acid bacteria include Lactobacillus plantarum, which degrades specific peptide bonds in gluten, improves the intestinal environment, and is effective for degrading gluten components, Lactobacillus acidophilus, which works with digestive enzymes to aid the digestion of proteins like gluten, and maintains the balance of beneficial intestinal bacteria, providing beneficial effects for intestinal health, Bifidobacterium breve, which produces gluten-degrading enzymes, helping to break down gluten proteins and contributing to reducing the digestive burden for gluten-sensitive individuals, and Lactobacillus rhamnosus, which reduces the immune response to gluten, improves the intestinal environment, and helps lessen the impact of gluten components on the intestinal barrier.

Gluten-degrading lactic acid bacteria reduce digestive burden by hydrolyzing the protein chains of gluten, thereby degrading gluten peptides and breaking gluten down into smaller amino acids and peptides, and they also degrade gliadin, a main component of gluten, which reduces harmful effects on the intestine and alleviates symptoms of gluten intolerance.

Lactic acid produced by gluten-degrading lactic acid bacteria offers the advantage of lowering intestinal pH, which inhibits the growth of harmful bacteria and provides an environment where beneficial bacteria may thrive, and it also protects the intestinal mucosa and reduces intestinal permeability.

Gluten-degrading lactic acid bacteria possess enzymes that convert polyphenols within the body, which contributes to suppressing inflammation, reducing inflammation of the intestinal wall, and is effective for preventing inflammatory bowel diseases like enteritis.

Gluten-degrading lactic acid bacteria synthesize B vitamins within the intestine, which is effective for energy metabolism, maintaining intestinal health, promoting metabolic activity, promoting the regeneration of intestinal mucosal cells, recovering damaged intestinal barrier, and protecting damaged intestinal barrier.

Gluten-degrading lactic acid bacteria improve the body's absorption rate of amino acids produced during gluten digestion, which may lead to enhanced intestinal mucosa regeneration and strengthened immunity due to the amino acids.

Gluten-degrading lactic acid bacteria offer the advantage of exhibiting a comprehensive anti-aging effect through their contributions to intestinal health, suppression of inflammatory responses, strengthening of intestinal immunity, and promotion of antioxidant substance generation within the intestine.

The enzyme food 100 includes 29 to 40 parts by weight of the enzyme preparation per 100 parts by weight of the enzyme food 100, and to maximize the efficacy derived from the enzyme preparation, it preferably includes 38.8 parts by weight of the enzyme preparation.

The powder preparation may be digestion-resistant maltodextrin, Bacillus subtilis powder, Aspergillus oryzae powder, cereal mixture powder, or germinated brown rice.

In addition, the powder preparation may be a combination of digestion-resistant maltodextrin, Bacillus subtilis powder, Aspergillus oryzae powder, cereal mixture powder, and germinated brown rice.

Digestion-resistant maltodextrin refers to maltodextrin that resists digestion and absorption in the body, and digestion-resistant maltodextrin that has not been fully digested in the small intestine moves to the large intestine, where approximately 50% thereof is fermented and then consumed serves as a food source for beneficial intestinal bacteria, and helps the growth of lactic acid bacteria, thereby improving the intestinal environment.

In addition, multiple studies have confirmed that digestion-resistant maltodextrin is slowly digested and absorbed in the body, lowers fasting blood sugar, and suppresses post-meal blood sugar surges, and digestion-resistant maltodextrin offers the advantage of helping to prevent diabetes.

In addition, due to its slow digestion property, digestion-resistant maltodextrin slows the rate of sugar absorption into the bloodstream, and prevents rapid insulin spikes, and lower insulin levels, in turn, reduce the accumulation of blood triglycerides, which offers the advantage of helping to prevent vascular disease, arteriosclerosis, hypertension, myocardial infarction, and stroke.

Bacillus subtilis, also known as hay bacillus, primarily resides in soil and belongs to the genus Bacillus, and is a spore-forming, gram-positive bacterium that tests positive in a gram stain reaction.

Bacillus subtilis is found among the microorganisms involved in fermenting Cheonggukjang (fast-fermented soybean paste) and Doenjang (soybean paste), and possesses strong vitality, enabling survival even in environments exceeding 150° C., and may reach the large intestine, making it effective for aiding bowel movements.

Bacillus subtilis powder is manufactured via a preparation operation involving preparing a Bacillus subtilis strain, a culturing operation involving culturing the strain in a medium supplied with nutrients such as glucose and protein, a concentration operation involving concentrating the fermented culture medium, a centrifugation operation involving centrifuging the concentrated medium to collect Bacillus subtilis cells and remove unnecessary medium components, and a powdering operation involving drying the Bacillus subtilis cells to produce powder.

Aspergillus oryzae, used for Aspergillus oryzae powder, is a mold utilized in manufacturing traditional fermented foods in Japan and Korea, such as Doenjang, soy sauce, Gochujang, and Cheongju, and in Japan, it is known as Koji, and during solid fermentation, it produces enzymes that break down starch and protein, thereby enhancing food flavor and promoting digestion and absorption.

When Aspergillus oryzae powder is ingested, amylase, one of the enzymes produced by Aspergillus oryzae during fermentation, degrades starch by converting it into smaller molecules such as glucose and dextrin, and breaks down carbohydrates into sugars, thereby promoting digestion.

Protease, another enzyme produced by Aspergillus oryzae, breaks down proteins into amino acids and small peptides, and these resulting amino acids aid in the regeneration of muscles and tissues and help strengthen immunity.

Lipase, another enzyme produced by Aspergillus oryzae, breaks down fats into fatty acids and glycerol, and it is effective for improving cholesterol levels by promoting fat metabolism.

Glucoamylase, another enzyme produced by Aspergillus oryzae, breaks down complex carbohydrates, converting them to glucose, and helps prevent rapid blood sugar spikes because its conversion rate to glucose is slow.

Vitamins B1, B2, B3, and B6, produced during a fermentation process by Aspergillus oryzae, support nerve and muscle function and promote energy metabolism, thereby contributing to fatigue recovery and enhanced physical vitality.

Antioxidant components produced by Aspergillus oryzae offer the advantage of supporting the immune system, preventing cell damage and aging by removing oxygen free radicals, and suppressing inflammatory responses within the body.

Cereal mixture powder is manufactured by grinding and mixing barley, glutinous rice, white soybean, wheat, brown rice, sorghum, foxtail millet, corn, perilla seeds, small black soybeans, adlay, peanuts, yam, proso millet, Seoritae (black soybeans harvested after the first frost comes down), peas, black rice, sesame seeds, black sesame seeds, and non-glutinous rice.

Included in the cereal mixture powder, Barley, glutinous rice, white soybean, wheat, brown rice, sorghum, foxtail millet, corn, proso millet, and non-glutinous rice serve as sources of carbohydrates and complex carbohydrates used for energy in the body, and adlay, perilla seeds, small black soybeans, and black rice are rich in protein and carbohydrates, providing satiety and sustained energy.

Each grain included in the cereal mixture powder contains vitamins such as B1, B2, and B3, which promote energy metabolism and contribute to fatigue recovery and vitality enhancement, and brown rice, barley, sorghum, and foxtail millet are rich in insoluble and soluble dietary fiber, aiding digestion and stimulating intestinal peristalsis, making them effective for preventing constipation.

Perilla seeds, sesame seeds, black sesame seeds, and peanuts contain abundant unsaturated fatty acids, such as omega fatty acids, which improve cholesterol levels and promote blood circulation, and small black soybeans, adlay, and glutinous rice are rich in magnesium and potassium, which regulate blood pressure and are effective for improving cardiovascular health.

Black rice, black sesame seeds, and Seoritae contain antioxidant components like anthocyanins, which remove oxygen free radicals, prevent cell damage, and strengthen immunity, and peanuts, sesame seeds, and black sesame seeds are rich in vitamin E and selenium, which prevent aging and activate immune cell function, thereby enhancing overall immunity.

Adlay, black sesame seeds, sesame seeds, and small black soybeans are rich in calcium and phosphorus, which strengthen bone density and are effective for preventing osteoporosis, and protein contained in white soybeans, Seoritae, and peas strengthens bones and muscles and is effective for maintaining joint health.

Brown rice, barley, sorghum, foxtail millet, and proso millet contain complex carbohydrates that slowly raise blood sugar, thus aiding in blood sugar control, and black rice, perilla seeds, sesame seeds, and black sesame seeds contain abundant antioxidants, making them effective for preventing skin aging and improving skin health.

White soybeans, Seoritae, small black soybeans, and peas provide high-quality protein containing essential amino acids, supporting muscle formation and recovery, and they also contain vitamin B6, which is necessary for protein metabolism and aids in muscle recovery.

Germinated brown rice powder is made from rice that has been sprouted from brown rice with the husk removed, to a length of about 1 to 5 mm, and compared to brown rice, it is relatively easier to digest and is rich in γ-aminobutyric acid (GABA), γ-oryzanol, and octacosanol, which are effective for providing anti-cancer benefits and reducing cholesterol levels in the body.

γ-aminobutyric acid acts as a neurotransmitter, inhibiting brain excitation and producing a calming effect, which helps relieve stress and reduce anxiety, and it also contributes to lowering blood pressure by relaxing blood vessels, making it effective for managing hypertension.

Soluble dietary fiber contained in germinated brown rice helps the growth of beneficial intestinal bacteria and promotes digestion, while insoluble dietary fiber contained in germinated brown rice promotes intestinal peristalsis, thereby preventing constipation.

Antioxidant components contained in germinated brown rice, including ferulic acid, phytic acid, and α-tocopherol, prevent cell damage by neutralizing free radicals, and also suppress aging-related cell damage and strengthen immunity by promoting immune cell activity.

B vitamins contained in germinated brown rice, such as vitamin B1 (thiamine), vitamin B2 (riboflavin), and vitamin B3 (niacin), support energy metabolism and aid in fatigue recovery, and vitamin B1 plays an important role in nerve health and muscle function, while vitamin B2 helps maintain skin health.

Essential amino acids contained in germinated brown rice contribute to muscle formation, tissue recovery, strengthening immunity, and fatigue recovery, and oryzanol, an antioxidant component contained in germinated brown rice, is effective for inhibiting cholesterol absorption in the body and lowering low-density lipoprotein cholesterol levels.

The enzyme food 100 includes 26 to 37 parts by weight of the powder preparation per 100 parts by weight of the enzyme food 100, and to best improve the efficacy and taste preference derived from the powder preparation, it preferably includes 35.22 parts by weight of the powder preparation.

Apple powder is obtained by drying apple juice, and a method of manufacturing apple powder includes a washing operation involving collecting and washing apples, an extraction operation involving slicing the apples, removing seeds, and then extracting juice from the apples along with the peel by using a juicer or extractor, a concentration operation involving concentrating the extracted apple juice at a low temperature, and a powdering operation involving obtaining powder from the apple juice via spray drying or freeze drying.

Apples, used as the ingredient for apple powder, contain pectin, which is a soluble dietary fiber effective for improving constipation, and apples also have properties that help make the skin clear and fair. Furthermore, consuming apples with the peel allows for ingestion of insoluble fiber, which is effective for aiding the movement of food through the digestive tract and preventing constipation.

In addition, pectin promotes the growth of beneficial intestinal bacteria and contributes to maintaining intestinal health, and also offers the advantage of helping to stabilize blood sugar and lower low-density lipoprotein cholesterol levels.

Soluble fiber contained in apples helps prevent cholesterol accumulation on blood vessel walls, thereby lowering the incidence of arteriosclerosis and heart disease, and vitamin C enhances the body's immunity, blocks cell damage, strengthens the epithelial barrier against pathogens, and improves immune system functions, including protection from various stresses.

Potassium contained in apples helps reduce blood pressure by promoting the excretion of unnecessary sodium, the pectin component in apples helps prevent diabetes by absorbing carbohydrates, and citric acid in apples offers the advantage of reducing fatigue, shoulder stiffness, and back pain.

Dietary fiber contained in apples reduces constipation and lowers the risk of colorectal cancer. As a high-fiber food, apples increase post-meal satiety, making them effective for dieting, and apple peel contains anthocyanins, which provide a strong antioxidant effect.

Tannins contained in apples aid physiological regulation and help generate brain neurotransmitters, thereby improving memory and helping to prevent dementia, and quercetin contained in apples helps protect the lungs by detoxifying nicotine within the body. In addition, organic acids in apples offer the advantage of helping to prevent anemia by aiding iron absorption.

Polyphenols contained in apples include flavonoids and chlorogenic acid. Flavonoids exhibit excellent antioxidant effects. Quercetin, kaempferol, and epicatechin are contained in apples.

Flavonoids contained in apples help neutralize oxygen free radicals within the body, thereby preventing cell damage, reducing inflammation, and helping to lower the risk of heart disease and cancer, and they also offer the advantage of preventing aging.

Chlorogenic acid contained in apples primarily helps regulate blood sugar levels by inhibiting gluconeogenesis in the liver, and it is effective for diabetes management when consumed by individuals with diabetes.

Cellulose, an insoluble dietary fiber contained in apples, is effective for promoting intestinal movement and preventing constipation, and vitamin C contained in apples enhances immunity and promotes collagen synthesis, thereby maintaining healthy skin and cartilage tissue.

Vitamin E contained in apples is a fat-soluble antioxidant that protects cell membranes and promotes skin health, and potassium contained in apples plays an important role in regulating blood pressure and supporting nerve and muscle function, offering a particular benefit for individuals with hypertension.

Malic acid and citric acid, which are organic acids contained in apples, offer the advantage of promoting digestion and helping fatigue recovery, and malic acid offers the advantages of activating liver function and promoting metabolism.

Carotenoids, antioxidant components contained in apples, particularly in the peel, offer the advantage of supporting eye health and providing benefits to the skin and immune system upon ingestion.

Triterpenoids contained in apples, particularly in the peel, offer the advantage of inhibiting cancer cell growth upon ingestion, notably attacking liver, colon, and breast cancer cells.

Procyanidins contained in apples, upon absorption into the body, help dilate blood vessels and lower blood pressure, and play an important role in cardiovascular health and offer the advantage of helping to improve blood circulation.

Therefore, consumption of the enzyme food 100 containing apple powder may provide the above-described benefits associated with apples. Furthermore, because the powder is manufactured by using juice extracted from apples including their peels via a juicer or extractor, the enzyme food 100 advantageously includes the benefits of the apple peel as well.

The enzyme food 100 includes 8 to 13 parts by weight of apple powder per 100 parts by weight of the enzyme food 100, and to best improve the efficacy and taste preference derived from the apple powder, it preferably includes 11.4 parts by weight of the apple powder.

Dextrin is a general term for low-molecular-weight carbohydrates, and refers to maltodextrin, which consists of at least three glucose units and emerges as an intermediate product during the digestive breakdown of carbohydrates, ultimately yielding glucose upon final breakdown.

As a polysaccharide, dextrin is easily digested and absorbed into the body as quickly as glucose, and in various food products, including enzyme foods, it is used as a thickener or filler and helps to increase shelf life.

Being easily digested and rapidly broken down in the body, dextrin serves as an energy source, and is useful for quickly supplying energy after exercise or for replenishing energy during prolonged activities.

Dextrin exhibits prebiotic functions, serving as food for beneficial intestinal bacteria and thus supporting intestinal health, and accordingly, it offers the advantage of improving the intestinal environment and promoting overall digestive health.

Dextrin has a relatively low glycemic index and helps to raise blood sugar levels slowly, making it useful for individuals requiring post-meal blood sugar management. By breaking down slowly in the body, it contributes to reducing blood sugar spikes.

When mixed with water, dextrin becomes viscous, which may contribute to a feeling of satiety, and in particular, digestion-resistant dextrin, which is rich in dietary fiber, slows the digestion rate, thereby helping to maintain satiety for longer periods and aiding in dieting efforts.

Dextrin contributes to lowering blood cholesterol levels by inhibiting cholesterol absorption in the intestine and positively impacts cardiovascular health. Due to these various benefits, it is widely used in products such as enzyme foods, nutritional supplements, sports drinks, and diet foods.

When absorbed in the body, dextrin may have the effect of preventing colorectal cancer. Furthermore, because it positively influences overall digestion, it offers the advantage of potentially increasing the body's absorption rate of other ingredients, including the enzyme preparation and the powder preparation.

Dextrin is an ingredient typically obtained by subjecting starch from corn, rice, potatoes, and the lie, to heating, acid treatment, or enzyme treatment. A specific method of manufacturing dextrin includes a starch preparation operation involving dissolving starch in water to prepare a slurry, an acid hydrolysis operation involving adding acid to hydrolyze the starch, so as to generate a first dextrin with short chains, a heat treatment operation involving heating the first dextrin and modifying its solubility, color, and viscosity to produce a second dextrin, and a drying and powdering operation involving drying and powderizing the second dextrin into a final product.

In the method of manufacturing dextrin, the acid hydrolysis operation may be replaced with an enzyme treatment operation involving partially decomposing the starch by using specific enzymes such as amylase, to generate the first dextrin.

Dextrin offers the advantage of exhibiting a comprehensive anti-aging effect through its contributions to increasing beneficial intestinal bacteria, providing antioxidant effects, exerting anti-inflammatory effects, supporting cardiovascular health, improving constipation, removing toxins, and aiding weight management.

The enzyme food 100 includes 5 to 9 parts by weight of dextrin per 100 parts by weight of the enzyme food 100, and to maximize the efficacy derived from dextrin, it preferably includes 7.2 parts by weight of dextrin.

Fructo-oligosaccharides occur naturally, primarily in fruits and vegetables, and recently, they have been used as a key component in enzyme foods and are generally manufactured via enzymatic or microbial reactions.

In detail, fructo-oligosaccharides are manufactured via a preparation operation involving preparing natural sugars obtained from sugar beet and sugarcane, an enzyme treatment operation where enzymes including a fructosyltransferase are added to link monosaccharide fructose molecules together in chains, to produce fructo-oligosaccharides in which fructoses are linked, and a separation and purification operation where the produced fructo-oligosaccharides are separated from other byproducts and purified to yield a high-purity product.

Fructo-oligosaccharides are not broken down by stomach acid and, unlike sugar, are not hydrolyzed by digestive enzymes in the small intestine, thus allowing them to reach the large intestine. Research findings to date indicate that approximately 90% of ingested fructo-oligosaccharides are found in the large intestine.

As prebiotics, fructo-oligosaccharides serve as food for beneficial bacteria in the large intestine, including Bifidobacteria (Bifidus), and promotes the growth of beneficial intestinal bacteria, thereby improving digestive function.

Fructo-oligosaccharides are known to exhibit relatively better effects than inulin or dextrin regarding enzyme induction and increasing Bifidus populations, and they also offer the advantage of preventing constipation through effects such as increasing intestinal peristalsis, increasing intestinal water content, and shortening intestinal transit time.

Fructo-oligosaccharides provide approximately 1 to 2 kcal/g, whereas sugars and carbohydrates typically provide 4 kcal/g, and thus, fructo-oligosaccharides are useful for limiting the intake of high-calorie foods and aiding in dieting.

Because fructo-oligosaccharides are not broken down by enzymes in the small intestine, they do not require insulin for metabolism, and as they are not absorbed into the bloodstream, they do not cause a rise in blood sugar levels, making their consumption possible for individuals with diabetes.

Fructo-oligosaccharides increase beneficial intestinal bacteria, which helps maintain a healthy intestinal barrier and plays an important role in strengthening the immune system, and they also offer the advantage of positively influencing immunity enhancement.

Fructo-oligosaccharide are slowly digested in the body, and thus are effective for reducing blood sugar spikes. Furthermore, according to some studies, consumption of fructo-oligosaccharides may contribute to lowering blood cholesterol levels.

Fructo-oligosaccharides reduce blood low-density lipoprotein (LDL)-cholesterol levels but do not lower blood high-density lipoprotein (HDL) cholesterol levels, offering the advantage of being effective against arteriosclerosis.

Fructo-oligosaccharides acidify the intestinal environment, which helps the absorption of minerals like calcium. By promoting the absorption of calcium, important for bone health, they offer the beneficial advantage of aiding in osteoporosis prevention. Fructo-oligosaccharides are widely utilized in enzyme foods, dairy products, and beverages.

Fructo-oligosaccharides possess effects such as improving lipid metabolism, providing anti-obesity benefits, preventing cardiovascular disease, and reducing triglyceride synthesis, and they also promote the absorption of minerals, including calcium and magnesium, and help prevent tooth decay.

Fructo-oligosaccharides offer the advantage of comprehensively slowing the aging process through contributions such as promoting beneficial bacteria growth, inhibiting harmful bacteria, providing anti-inflammatory effects, improving immunity, promoting antioxidant substance production, reducing calorie intake, and enhancing intestinal health.

The enzyme food 100 includes 2 to 5 parts by weight of fructo-oligosaccharides per 100 parts by weight of the enzyme food 100, and to maximize the efficacy derived from fructo-oligosaccharides, it preferably includes 3.1 parts by weight of fructo-oligosaccharides.

Isolated soy protein is protein extracted from soybeans. The solid residue remaining after extracting soybean oil from soybeans by using the organic solvent hexane is referred to as defatted soybeans, and isolated soy protein is manufactured by removing non-protein components, such as soluble and insoluble carbohydrates, from defatted soybeans.

Isolated soy protein promotes adiponectin secretion, thereby aiding dieting efforts, and also lowers blood cholesterol levels and improves vascular health. Through continuous consumption, it offers the advantage of helping to prevent cardiovascular diseases.

Isolated soy protein promotes the secretion of adiponectin, which is a hormone produced by fat cells. Because adiponectin acts to break down body fat, periodically consuming isolated soy protein offers the advantage of reducing body fat or abdominal fat.

Isolated soy protein may be used in carbohydrate-predominant diets to increase protein intake, and thus, it offers the advantages of preventing the deterioration of hair and nail condition caused by protein deficiency, increasing immunity, and helping to prevent the occurrence of anemia.

Solae, a company leading research on isolated soy protein, submitted a petition in 2004 to the U.S. Food and Drug Administration (FDA) seeking approval for a health claim stating that isolated soy protein is effective in inhibiting breast, prostate, and colon cancer. According to Solae's research findings, consumption of isolated soy protein was associated with a reduction in breast and prostate cancer.

Isoflavones, saponins, amino acids, and genistein contained in isolated soy protein help suppress stress and strengthen immunity, offering the advantage of inhibiting the occurrence of cancer and the progression of disease.

Genistein contained in isolated soy protein inhibits oxidation, which is a chemical reaction that damages intracellular deoxyribonucleic acid (DNA) and may cause cancer, and helps prevent the occurrence of cancer by enhancing the body's immunity.

Phytic acid contained in isolated soy protein has an excellent effect against conditions like colorectal cancer by blocking the absorption of minerals such as iron, which may otherwise cause oxidation when absorbed into the body.

Isolated soy protein inhibits angiogenesis, which is the formation of new blood vessels, when cancer tumors develop, thereby reducing blood flow to the tumors, and offers the advantage of reducing tumor size and latency period.

In high-stress situations, the body's cells produce stress proteins that go against the normal cell cycle, and such a stressful condition may become a cause of cancer development, allowing cancer cells to continuously proliferate.

Genistein contained in isolated soy protein inhibits the production of stress proteins by cells that go against the normal cell cycle, and this inhibition of stress protein production offers the advantage of suppressing cancer development.

Daidzein contained in isolated soy protein helps strengthen the body's immunity, which is closely linked to the occurrence and progression of cancer, and together with genistein, it offers the advantage of effectively inhibiting cancer development.

Genistein contained in isolated soy protein inhibits the production of tyrosine kinases, which are enzymes essential for the development of certain cancers and the proliferation of cancer cells, thereby reducing cancer development and cancer cell proliferation.

In general, osteoporosis is a disease characterized by frequent fractures as bones weaken due to lowered bone mineral density (BMD), and because it often progresses silently, awareness of its progression is difficult until a bone fracture occurs.

When isoflavones contained in isolated soy protein are consumed periodically, bone mineral density tends to increase in proportion to the amount of isoflavones consumed, which offers the advantage of being effective for preventing osteoporosis.

Climacteric syndrome in menopausal women causes problems related to the autonomic nervous system, mental state, joints, and digestive system. Facial flushing, one symptom occurring early in menopause, appears at a relatively higher rate in European women compared to Asian women.

Genistein, daidzein, and glycitein, contained in isolated soy protein, act very similarly to estrogen when absorbed into the body, and they offer the advantage of inhibiting the occurrence of climacteric syndrome in menopausal women.

Antioxidant components contained in isolated soy protein remove reactive oxygen species that cause cell damage, amino acids contained in isolated soy protein play an important role in promoting collagen synthesis, anti-inflammatory components contained in isolated soy protein reduce inflammatory responses, isoflavones contained in isolated soy protein help maintain hormone balance, and high-quality proteins contained in isolated soy protein maintains or increases muscle mass. Consequently, isolated soy protein offers the advantage of high anti-aging efficacy.

The enzyme food 100 includes 1 to 4 parts by weight of isolated soy protein per 100 parts by weight of the enzyme food 100, and to maximize the efficacy derived from isolated soy protein, it preferably includes 2.1 parts by weight of isolated soy protein.

The synthetic flavoring agent is an ingredient used to artificially impart aroma. Considering apple powder, vinegar powder, and DL-malic acid used in the enzyme food 100, it is preferable to use a synthetic apple flavoring agent that provides an apple aroma.

To provide an apple aroma while maximizing the intestinal health benefits of the enzyme food 100, the synthetic flavoring agent may include ethyl hexanoate, hexyl acetate, butyl acetate, hexenal, linalool, and methyl butyrate, which place a low burden on the intestine and indirectly support intestinal health.

Ethyl Hexanoate is a representative synthetic compound providing the sweet and fresh aroma of apples, and is well-digested and easily metabolized in the body, offering the advantage of placing a low burden on the intestine.

Hexyl acetate is a synthetic flavoring used to reproduce apple and pear aromas, and exhibits no toxicity or harmful intestinal reactions and is easily digested and absorbed. Butyl acetate is a component providing a sweet fruit aroma, used in various fruit flavorings including apple, and is well-digested.

Hexenal provides the aroma of fresh apples and is a component extracted from plant-based ingredients to be used to reproduce the apple scent. It is known to possess antioxidant activity, thereby indirectly contributing to reducing intestinal inflammation.

Linalool is a component used in various fruit and flower aromas and is employed to create the complex aroma of apples. It possesses anti-inflammatory effects, helping to reduce intestinal inflammation, and also offers the advantage of being effective for stress relief.

Methyl butyrate is used to reproduce fresh apple and strawberry aromas and to provide a refreshing, sweet scent. It is a compound that does not place a significant burden on digestion and is considered a safe ingredient that does not burden the intestinal environment.

The enzyme food 100 includes 0.375 to 2 parts by weight of a synthetic flavoring agent per 100 parts by weight of the enzyme food 100, and to best improve the efficacy and taste preference derived from the synthetic flavoring agent, it preferably includes 0.75 parts by weight of the synthetic flavoring agent.

The amino acid mixture powder includes 45 to 55 parts by weight of L-leucine, 22.5 to 27.5 parts by weight of L-isoleucine, 22.5 to 27.5 parts by weight of L-valine, and sunflower lecithin.

L-leucine (C₆H₁₃NO₂) is one of the essential amino acids that constitute proteins, and because it is not synthesized in the body, it must be obtained through diet. It plays an important role in protein synthesis, tissue repair, and energy production.

L-leucine activates the mammalian target of rapamycin (mTOR) pathway, thereby promoting protein synthesis. mTOR is a crucial signaling pathway for cell growth and protein synthesis, and activating this pathway stimulates muscle cells to synthesize more protein, which is essential for muscle growth.

L-leucine inhibits protein breakdown within muscles, which helps prevent muscle loss in the elderly or individuals recovering from illness. It is also used as an energy source via gluconeogenesis.

L-leucine plays a role in replenishing energy when the body is in an energy deficit state. During exercise, L-leucine may be used as an energy source, offering the advantage of reducing physical fatigue and improving endurance.

Insulin lowers blood sugar and enables cells to use blood glucose as an energy source. L-leucine promotes insulin secretion, which helps regulate blood sugar levels, and also offers the advantage of aiding body fat reduction by promoting fat oxidation.

L-isoleucine (C₆H₁₃NO₂) is one of the essential amino acids, and because it is not synthesized in the body, it must be obtained through diet or supplements. It plays an important role in muscle tissue recovery, energy production, and endurance maintenance.

L-isoleucine is utilized as an energy source within muscles and plays an important role in helping muscle tissue generate glucose needed for energy metabolism. It aids in reducing fatigue and enhancing endurance.

L-isoleucine helps regulate blood sugar levels and stimulates insulin secretion, thereby helping cells utilize blood sugar for energy. It also promotes the generation of white blood cells and helps strengthen the immune response, increasing resistance to infections and diseases.

L-isoleucine helps prevent muscle tissue damage and promotes recovery. It is necessary for rapid muscle recovery after strenuous exercise and contributes to maintaining muscle mass by inhibiting muscle protein breakdown.

L-isoleucine plays an important role in hemoglobin generation, and via specific enzymes, it is converted into intermediate metabolites such as ketoglutarate and succinate. Adenosine triphosphate (ATP), generated during this conversion process, provides immediate energy required during exercise. Furthermore, the unique structure of L-isoleucine contributes to blood sugar regulation and fat oxidation.

L-valine is one of the essential amino acids, and because it is not synthesized in the body, it must be obtained through diet or supplements. It plays an important role in muscle metabolism, tissue repair, and energy production.

L-valine, along with L-leucine and L-isoleucine, belongs to the group of branched-chain amino acids (BCAAs). These three amino acids are effective for muscle growth, recovery, and enhancing endurance.

L-valine plays an important role in supplying energy to muscles during exercise, and may be used as an alternative energy source when glucose is deficient, thus contributing to reducing fatigue during exercise. Converted into an energy source in the liver, it supports muscle activity and improves the body's endurance.

L-valine promotes muscle protein synthesis, thereby supporting muscle growth, and also inhibits protein breakdown, helping to maintain muscle tissue. Furthermore, it regulates serotonin levels, which helps stabilize mood and reduce stress or anxiety.

L-valine promotes white blood cell generation, thereby strengthening immunity, and is effective for preventing muscle damage and promoting tissue recovery. It also plays an important role in gluconeogenesis, helping muscles continuously receive the energy they need.

Sunflower lecithin is a lecithin extracted from sunflower seeds and is a type of natural plant-based phospholipid. As a component constituting cell membranes, it plays an important role in maintaining cell structure, nerve function, and metabolism.

Phosphatidylcholine and linoleic acid, contained in sunflower lecithin, help lower blood low-density lipoprotein cholesterol levels and increase beneficial high-density lipoprotein cholesterol levels, which helps maintain artery health and prevent cardiovascular diseases.

Phosphatidylcholine contained in sunflower lecithin is a primary source of choline and is converted within the body into the neurotransmitter acetylcholine. Acetylcholine enhances memory and cognitive function, thereby aiding in the improvement of brain health, concentration, and memory.

Phosphatidylcholine contained in sunflower lecithin is an important component of liver cell membranes, and contributes to breaking down and eliminating fat accumulated in the liver, making it effective for liver health and detoxification.

Sunflower lecithin acts as an emulsifier within the digestive tract, promoting the effective mixing of fats and water. This, in turn, promotes intestinal fat digestion and reduces the burden on the digestive system.

Unsaturated fatty acids contained in sunflower lecithin strengthen the skin barrier, maintain moisture, and alleviate inflammation, which helps alleviate skin problems such as general skin trouble, dry skin, and eczema.

The enzyme food 100 includes 0.2 to 2 parts by weight of the amino acid mixture powder per 100 parts by weight of the enzyme food 100, and to maximize the efficacy derived from the amino acid mixture powder, it preferably includes 0.4 parts by weight of the amino acid mixture powder.

Licorice concentrate powder is an ingredient in a powder form, manufactured by drying a licorice extract. Licorice exhibits excellent therapeutic effects for general inflammation, gastritis, and stomatitis, and is effective in treating sore throat, infectious hepatitis, skin eczema, acne, and peptic ulcers, and aiding detoxification.

Licorice, used for manufacturing the licorice concentrate powder, is a perennial plant belonging to the legume family. It is native to Asia and grows wild or is cultivated in regions including Northern China, Manchuria, Mongolia, Siberia, and Southern Italy.

The reddish-brown root of licorice remains inserted deep underground, and the stem exhibits a sweet taste, so when added to bitter foods, it neutralizes the bitterness and improves the palatability or preference for the food.

Licorice, used for manufacturing the licorice concentrate powder, contains glycyrrhizin, glabric acid, sucrose, glucose, liquiritin, and liquiritigenin, which are sweetening components.

Glycyrrhizin is a type of saponin glycoside, and upon decomposition, produces glucuronic acid, which combines with toxic substances in the liver to perform detoxification. Consequently, it helps restore liver function, and may also enable the treatment of drug addiction, hepatitis, urticaria, dermatitis, and eczema.

In traditional Korean medicine, licorice is known for neutralizing the toxicity of various medicines, thereby harmonizing their effects. The Bencao gangmu (Compendium of Materia Medica) discloses that licorice strengthens muscles and bones, promotes the growth of new flesh, and boosts vitality.

Glycyrrhizin contained in licorice possesses an anti-inflammatory function that inhibits inflammation. This helps alleviate inflammatory diseases such as skin inflammation, asthma, and arthritis, and offers the advantage of helping to soothe sensitive skin.

Glycyrrhizin contained in licorice inhibits viral proliferation and exhibits an antiviral effect that strengthens the immune system, thereby inhibiting the proliferation of the herpes virus and hepatitis virus. In some countries, it is used as an adjuvant therapeutic agent for hepatitis.

Glycyrrhizin contained in licorice modulates immune cell activity, thereby maintaining immune balance and suppressing excessive immune responses, and it has been researched as an adjuvant for autoimmune diseases or conditions of compromised immunity.

Glycyrrhizin contained in licorice helps protect liver cells and restore damaged liver function, is effective for removing toxins from the liver via its antioxidant action, and prevents bodily aging through strong antioxidant and anti-aging effects.

Glabric acid, a flavonoid component extracted from licorice root, is effective for skin whitening by inhibiting melanin production, and alleviates pigmentation caused by ultraviolet rays.

As a strong antioxidant, glabric acid contained in licorice inhibits the oxidation of skin cells, thus exhibiting anti-aging and cell-protective effects. Through its anti-inflammatory function, glabric acid reduces inflammation and helps with sensitive skin or inflammatory skin conditions.

Glabric acid contained in licorice exhibits antimicrobial and antiviral actions, inhibiting the growth of bacteria and viruses, and also offers the advantages of regulating blood pressure and lowering cholesterol levels.

Glucose contained in licorice is the most basic monosaccharide and functions importantly as an energy source, and is an essential energy source for a human body and possesses various physiological benefits.

Glucose contained in licorice plays an important role in regulating blood sugar levels via insulin, and is also effective for maintaining brain activity and cognitive function, as the brain uses glucose as its primary energy source.

Glucose contained in licorice aids in the production of various substances necessary for normal cell function and growth and plays an essential role in the body's metabolic processes. It also supplies energy that enables immune cells to function effectively, thus being effective for maintaining immunity.

Liquiritin contained in licorice inhibits melanin production, thereby alleviating skin pigmentation and helping to brighten skin tone, and also exhibits antioxidant effects, reducing oxidative stress in skin cells and preventing aging.

Liquiritin contained in licorice has the efficacy of inhibiting inflammation and soothing the skin, and thus offers the advantage of being effective through its anti-inflammatory and soothing actions, which alleviate sensitive skin or inflammatory skin conditions.

Liquiritigenin contained in licorice is effective for reducing cell damage and preventing aging due to its strong antioxidant function. Consequently, it maintains skin and is also beneficial for preventing various diseases.

Liquiritigenin contained in licorice is effective for inhibiting inflammatory responses and alleviating allergic reactions, and particularly offers the advantage of helping to alleviate skin inflammation or allergic diseases.

Liquiritigenin contained in licorice has the potential to inhibit the growth of certain cancer cells and thus is receiving attention in cancer prevention research. It also acts similarly to the female hormone estrogen, making it useful for regulating hormone balance.

Therefore, consuming the enzyme food 100 containing the licorice concentrate powder offers the advantage that the benefits of licorice, such as anti-inflammatory, anti-cancer, liver-protective, antioxidant, anti-aging, antiviral, and blood sugar control effects, may be expected.

The enzyme food 100 includes 0.15 to 2 parts by weight of the licorice concentrate powder per 100 parts by weight of the enzyme food 100, and to maximize the efficacy derived from the licorice concentrate powder, it preferably includes 0.3 parts by weight of the licorice concentrate powder.

Gluten-degrading lactic acid bacteria are lactic acid bacteria that degrade gluten, which is an insoluble protein responsible for gluten-sensitive enteropathy, gluten intolerance, gluten allergy, and flour addiction, and these bacteria offer the advantage of promoting the digestion of flour-based foods.

Gluten-degrading lactic acid bacteria include Lactobacillus plantarum, which degrades specific peptide bonds in gluten, improves the intestinal environment, and is effective for degrading gluten components, Lactobacillus acidophilus, which works with digestive enzymes to aid the digestion of proteins like gluten, and maintains the balance of beneficial intestinal bacteria, providing beneficial effects for intestinal health, Bifidobacterium breve, which produces gluten-degrading enzymes, helping to break down gluten proteins and contributing to reducing the digestive burden for gluten-sensitive individuals, and Lactobacillus rhamnosus, which reduces the immune response to gluten, improves the intestinal environment, and helps lessen the impact of gluten components on the intestinal barrier.

Gluten-degrading lactic acid bacteria reduce digestive burden by hydrolyzing the protein chains of gluten, thereby degrading gluten peptides and breaking gluten down into smaller amino acids and peptides, and they also degrade gliadin, a main component of gluten, which reduces harmful effects on the intestine and alleviates symptoms of gluten intolerance.

Lactic acid produced by gluten-degrading lactic acid bacteria offers the advantage of lowering intestinal pH, which inhibits the growth of harmful bacteria and provides an environment where beneficial bacteria may thrive, and it also protects the intestinal mucosa and reduces intestinal permeability.

Gluten-degrading lactic acid bacteria possess enzymes that convert polyphenols within the body, which contributes to suppressing inflammation, reducing inflammation of the intestinal wall, and is effective for preventing inflammatory bowel diseases like enteritis.

Gluten-degrading lactic acid bacteria synthesize B vitamins within the intestine, which is effective for energy metabolism, maintaining intestinal health, promoting metabolic activity, promoting the regeneration of intestinal mucosal cells, recovering damaged intestinal barrier, and protecting damaged intestinal barrier.

Gluten-degrading lactic acid bacteria improve the body's absorption rate of amino acids produced during gluten digestion, which may lead to enhanced intestinal mucosa regeneration and strengthened immunity due to the amino acids.

The enzyme food 100 includes 0.1 to 2 parts by weight of gluten-degrading lactic acid bacteria per 100 parts by weight of the enzyme food 100, and to maximize the efficacy derived from gluten-degrading lactic acid bacteria, it preferably includes 0.2 parts by weight of gluten-degrading lactic acid bacteria.

Guar gum is a natural polysaccharide extracted from plants, and is primarily obtained from guar beans. It is mainly used to increase the viscosity or enhance the stability of food products.

Guar gum is a soluble dietary fiber that absorbs moisture in the intestine, and convert the moisture into a gel-like substance. This softens the intestinal contents, making defecation easier, and offers the advantage of promoting intestinal movement.

Guar gum possesses prebiotic functions that promote the growth of beneficial intestinal bacteria, and functions related to managing blood sugar and cholesterol, and offers the advantage of lowering blood cholesterol levels by aiding the excretion of bile acids.

In detail, guar gum is composed of two main saccharides, galactose and mannose, and acts as food for beneficial intestinal bacteria, thereby improving the gut microbial environment. This offers the advantages of promoting digestive health and maintaining intestinal health.

The soluble dietary fiber in guar gum prevents the rapid absorption of glucose after meals, thereby delaying blood sugar spikes, and is useful for individuals who require blood sugar control and also aids in diabetes management.

Guar gum reduces cholesterol absorption in the intestine and is particularly effective for lowering low-density lipoprotein cholesterol levels. Accordingly, it is beneficial for preventing cardiovascular diseases and positively influences the improvement of hyperlipidemia.

By absorbing water and increasing in volume, guar gum induces satiety in the stomach, making it effective for reducing overeating and aiding weight control. The sustained feeling of satiety resulting from guar gum consumption offers the advantage of helping to control meal sizes and, particularly, manage appetite during dieting.

Guar gum serves as food for beneficial intestinal bacteria, enabling short-chain fatty acids produced by the beneficial intestinal bacteria to exert anti-inflammatory effects. This, in turn, helps reduce overall inflammation levels throughout the body, not just in the intestine.

Although guar gum itself does not contain direct antioxidant components, substances produced through its metabolism by gut microbiota contribute to antioxidant activity. Therefore, guar gum offers the advantage of being effective in preventing bodily aging.

The enzyme food 100 includes 0.05 to 2 parts by weight of guar gum per 100 parts by weight of the enzyme food 100, and to maximize the efficacy derived from guar gum, it preferably includes 0.1 parts by weight of guar gum.

In detail, the vinegar powder is an apple vinegar powder with mother of vinegar produced by drying apple vinegar with mother of vinegar, which is obtained by washing apples with peels remaining and seeds removed, placing the washed apples, sugar, and live bacteria into a sealed container, and then fermenting the mixture for a predetermined time period.

Through the fermentation process, the apple vinegar with mother of vinegar becomes enriched with beneficial components, and thus exhibits various benefits upon consumption. It is added to various enzyme foods or other food products to improve their taste and functionality.

The vinegar powder contains various organic acids, including fermented mother of vinegar, acetic acid, citric acid, tartaric acid, malic acid, and succinic acid. Acetic acid, a main component of apple vinegar with mother of vinegar, aids weight loss by preventing fat accumulation in the body, and is also effective for blood sugar control, slowing sugar absorption, and reducing insulin resistance.

Polyphenols contained in the vinegar powder are rich in antioxidant substances, and thus offer the advantages of improving skin health and preventing bodily aging by removing oxygen free radicals, and preventing cell damage.

Prebiotics, which are beneficial bacteria contained in the vinegar powder, upon consumption, promote the activity of beneficial intestinal bacteria and inhibit harmful intestinal bacteria, and help intestinal health and particularly offer the advantages of improving digestive health and strengthening immunity.

The vinegar powder functions as a natural preservative, protecting the body from Escherichia coli, bacteria, and viruses, thereby preventing food poisoning and stomach upsets, and also helps reduce abdominal fat and triglycerides.

The vinegar powder contains abundant organic acids and increases in acidity as it ferments into vinegar, allowing it to function as gastric acid, thereby promoting digestion. It also offers the advantages of increasing intestinal GLP-1 hormone secretion, which promotes insulin secretion, and increasing the AMPK enzyme, which enhances insulin sensitivity.

The vinegar powder contains minerals such as vitamin C, B vitamins, and potassium, which promote metabolism and activate fat burning. It helps with fatigue recovery and maintaining general health, lowers blood sugar levels, and reduces cholesterol and triglyceride (neutral fat) levels.

The vinegar powder is manufactured via a concentration process involving concentrating apple vinegar with mother of vinegar by evaporating moisture at low temperature, thereby reducing liquid volume while maintaining active ingredients, a freeze drying process involving rapidly freezing the concentrated apple vinegar with mother of vinegar and then drying it under vacuum, a grinding process involving grinding the freeze-dried apple vinegar with mother of vinegar by using a grinder, and then further grinding it into a fine powder, and a sealing process involving sealing the ground powder, protecting it from moisture and direct sunlight.

The enzyme food 100 includes 0.05 to 2 parts by weight of the vinegar powder per 100 parts by weight of the enzyme food 100, and to maximize the efficacy derived from the vinegar powder, it preferably includes 0.1 parts by weight of the vinegar powder.

The lactic acid bacteria mixture powder includes Lactobacillus plantarum, Lactobacillus rhamnosus, Lactobacillus casei, Lactobacillus acidophilus, Lactobacillus paracasei, Lactobacillus salivarius, Lactobacillus gasseri, Bifidobacterium breve, Lactobacillus helveticus, Streptococcus thermophilus, Bifidobacterium bifidum, Bifidobacterium longum, Bifidobacterium animalis subsp. lactis, Lactococcus lactis subsp. lactis, Lactobacillus fermentum, Lactobacillus reuteri, Lactobacillus bulgaricus, Bifidobacterium infantis, Leuconostoc citreum, Lactobacillus brevis, and Leuconostoc kimchii, and may further include dextrin.

In detail, the lactic acid bacteria mixture powder may include 17.87 parts by weight of Lactobacillus plantarum, 12.94 parts by weight of Lactobacillus rhamnosus, 8.77 parts by weight of Lactobacillus casei, 7.5 parts by weight of Lactobacillus acidophilus, 5.55 parts by weight of Lactobacillus paracasei, 5.23 parts by weight of Lactobacillus salivarius, 1.73 parts by weight of Lactobacillus gasseri, 1.5 parts by weight of Bifidobacterium breve, 1.25 parts by weight of Lactobacillus helveticus, 1.24 parts by weight of Streptococcus thermophilus, 1.22 parts by weight of Bifidobacterium bifidum, 1 part by weight of Bifidobacterium longum, 0.93 parts by weight of Bifidobacterium animalis subsp. lactis, 0.84 parts by weight of Lactococcus lactis subsp. lactis, 0.62 parts by weight of Lactobacillus fermentum, 0.5 parts by weight of Lactobacillus reuteri, 0.5 parts by weight of Lactobacillus bulgaricus, 0.005 parts by weight of Bifidobacterium infantis, 0.002 parts by weight of Leuconostoc citreum, 0.002 parts by weight of Lactobacillus brevis, 0.002 parts by weight of Leuconostoc kimchii, and 30.8 parts by weight of dextrin.

The microorganisms included in the lactic acid bacteria mixture powder are mostly probiotics, and when consumed, they balance beneficial intestinal bacteria and inhibit the growth of harmful bacteria, thereby promoting intestinal health. They also alleviate digestive problems such as constipation, diarrhea, and irritable bowel syndrome.

The microorganisms included in the lactic acid bacteria mixture powder help activate the immune system, increasing resistance to pathogenic microorganisms and strengthening defenses against infectious diseases, and they also offer the advantage of inhibiting intestinal inflammation.

The microorganisms included in the lactic acid bacteria mixture powder produce antimicrobial substances that inhibit harmful bacteria, making them effective for improving intestinal health, suppressing skin trouble, and preventing inflammatory diseases. They also promote digestion, which is effective for reducing intestinal gas and abdominal bloating.

The microorganisms included in the lactic acid bacteria mixture powder improve intestinal health and also improve skin health and positively affect mental health, both of which are closely linked to intestinal health. This offers the advantages of reducing stress and stabilizing mood.

The enzyme food 100 includes 0.05 to 2 parts by weight of the lactic acid bacteria mixture powder per 100 parts by weight of the enzyme food 100, and to best improve the efficacy and taste preference derived from the lactic acid bacteria mixture powder, it preferably includes 0.1 parts by weight of the lactic acid bacteria mixture powder.

Salt is an inorganic compound composed of sodium (Na) and chlorine (Cl). These chemical components play an important role in the body's physiological functions and possess various efficacies, including antimicrobial action, electrolyte function, and blood pressure regulation.

In detail, sodium plays a role in maintaining water balance within the body, balancing fluids inside and outside cells, and sodium ions perform an important function of maintaining cell volume by regulating osmotic pressure.

When a nerve cell receives a stimulus, sodium ions move across the cell membrane, generating an electrical signal, and nerve conduction occurs accordingly. Therefore, sodium is essential for delivering electrical signals between nerve cells.

Sodium, along with Potassium (K), aids in muscle contraction, and offers the advantage that sodium ions are involved in the contraction and relaxation processes of the heart muscle, thereby maintaining the heartbeat and facilitating smooth blood circulation.

Sodium is essential for maintaining the acid-base balance within the body. Blood hydrogen ion concentration index (pH) levels are regulated by sodium, offering the advantage that an optimal physiological environment is maintained for the body.

Chlorine is an essential component of gastric acid (HCl) produced in the stomach, and is vital for the digestion process. Gastric acid breaks down food, promotes the activity of digestive enzymes, and offers the advantage of inhibiting pathogenic bacteria within the stomach.

In addition, chloride ions, along with sodium and potassium ions, maintain the acid-base balance within the body. Through this, the hydrogen ion concentration index (pH) of blood and body fluids is kept constant. Chloride ions also offer the advantage of maintaining water balance by regulating osmotic pressure between cells and body fluids.

Salt offers the advantage of creating an environment difficult for microorganisms to survive, by rapidly changing water concentrations inside and outside cells in a way that causes dehydration of the microbial cell membrane, leading to cell destruction.

Salt acts as an electrolyte in the body, aiding electrical conduction, and is essential for maintaining nerve, muscle, and heart functions. An appropriate amount of sodium offers the advantage of keeping blood pressure stable.

Based on Korean standards, consuming 5 g or less of salt per day is considered appropriate. When consumed in appropriate amounts, salt helps regulate blood pressure and reduces the risk of cholesterol accumulation, making it effective for maintaining cardiovascular health.

Consuming an appropriate amount of salt helps maintain healthy inner walls of blood vessels, reduces the accumulation of low-density cholesterol in arteries, and helps maintain normal high-density cholesterol levels, thereby also improving the removal of low-density cholesterol from blood vessels.

Meanwhile, to avoid unintended effects beyond the desired physiological actions of salt, the enzyme food 100 may include refined salt, which lacks minerals, and may include, besides refined salt, bamboo salt or natural salt that is fortified with flavor and minerals.

The enzyme food 100 includes 0.05 to 2 parts by weight of salt per 100 parts by weight of the enzyme food 100, and to maximize the efficacy derived from salt, it preferably includes 0.1 parts by weight of salt.

The psyllium husk powder is an ingredient prepared in powder form from seed coats of psyllium. The psyllium husk powder is produced via a washing operation involving washing psyllium, a separation operation involving separating husk from the psyllium, a drying operation involving drying the separated husk by using a hot-air dryer or a natural drying method, and a grinding operation involving grinding the dried husk to produce a dietary fiber powder.

Plantago asiatica, the ingredient of the psyllium husk powder, is a dicotyledonous perennial plant belonging to the family Plantaginaceae and the order Plantaginales. The leaves of Plantago asiatica are referred to as ‘Chajeon’, and its seeds are referred to as ‘Chajeonja’.

Psyllium husk is composed mainly of soluble dietary fiber and thus has the property of absorbing water and swelling within the body, which improves intestinal health and is effective for blood sugar control.

In detail, psyllium husk is composed of hemicellulose and mucilage that are soluble dietary fibers, and because it transforms into a gel form upon combining with water, it slows the rate of digestion in the small intestine and inhibits the absorption of cholesterol and sugar. A small amount of insoluble dietary fiber present in psyllium husk promotes intestinal movement and contributes to constipation prevention.

Soluble dietary fiber contained in psyllium husk binds with cholesterol in the intestine and is then excreted from the body, thus offering the advantage of lowering low-molecular-weight cholesterol levels. It also slows the digestion and absorption of carbohydrates, which prevents rapid blood sugar spikes and aids in blood sugar management for individuals with diabetes.

Mucilage contained in psyllium husk transforms into a soft gel upon contact with water, increasing in volume and softening intestinal contents, and acts as a lubricant within the intestine, easing defecation and proving effective for preventing or improving constipation. Furthermore, by combining with food in the stomach, it helps maintain satiety for longer periods, thereby also aiding in weight control.

Hemicellulose contained in psyllium husk absorbs water in the intestine, thereby increasing stool volume, and also acts as food for beneficial bacteria within the intestine, improving the gut microbial environment and contributing to the growth of probiotics, which enhances intestinal health.

Protein and carbohydrates contained in psyllium husk improve the intestinal environment and prevent constipation. Furthermore, some carbohydrate components are fermented in the intestine, generating short-chain fatty acids that serve as food for beneficial gut bacteria, thus being effective for intestinal health.

Various antioxidant components contained in psyllium husk have the effect of inhibiting oxygen free radicals and reducing inflammation. As these antioxidant components play a role in reducing oxidative stress within the intestine, they are helpful for intestinal health.

The enzyme food 100 includes 0.05 to 2 parts by weight of the psyllium husk powder per 100 parts by weight of the enzyme food 100, and to maximize the efficacy derived from the psyllium husk powder, it preferably includes 0.1 parts by weight of the psyllium husk powder.

In addition, the psyllium husk powder included in the enzyme food 100 may be replaced with commercially available psyllium husk dietary fiber, and because the method of manufacturing psyllium husk dietary fiber is similar to the method of manufacturing a psyllium husk powder, their substantial components are identical.

Malic acid (C₄H₆O₅), a component contained in grapes or apples, possesses a sour taste similar to citric acid, and there are natural malic acid (L-malic acid) extracted from fruit, and synthetic malic acid (DL-malic acid) produced industrially via fermentation.

Malic acid acts as an intermediate product in the Krebs cycle or the tricarboxylic acid (TCA) cycle, which is an important process in cellular energy metabolism. Within this cycle, malic acid plays a crucial role in generating cellular energy, particularly aiding in ATP production.

By promoting energy production, malic acid helps in the body's fatigue recovery and stamina enhancement, and particularly offers the advantage of promoting energy metabolism by positively influencing post-exercise recovery.

Malic acid is effective in reducing oxidative stress and possesses the ability to neutralize reactive oxygen species (ROS). It influences the metabolic processes of antioxidant substances, reduces cell damage caused by free radicals, exhibits anti-aging and inflammation-relieving effects, and consequently also aids skin health.

Malic acid is classified as an alpha hydroxy acid (AHA) and possesses skin exfoliation and moisturizing functions. It offers the advantage of weakening bonds in the epidermal layer, which removes old dead skin cells and promotes new cell regeneration.

Malic acid promotes the secretion of digestive enzymes and gastric acid, thereby smoothing the digestion process. Furthermore, it positively influences intestinal microorganisms and aids the growth of lactic acid bacteria, proving effective for resolving gut-related issues such as improving indigestion and reducing abdominal bloating.

Malic acid binds with metals to perform a chelation action, thereby excreting heavy metals or toxic substances from the body, which contributes to improving liver function, reducing bodily toxins, and helping to maintain overall health and vitality.

Malic acid breaks down lactic acid, which causes pain and fatigue when it accumulates in muscles, thereby alleviating muscle pain, and is useful for reducing muscle fatigue and pain after strenuous exercise, and through this action, rapid physical recovery may be expected.

Malic acid promotes energy metabolism, helping to burn fat accumulated in the body, and offers the advantage that when body weight decreases, levels of low-density cholesterol and total cholesterol tend to naturally decrease as well.

Through its antioxidant action, malic acid may prevent the oxidation of cholesterol that promotes arteriosclerosis, by reducing oxidative stress on blood vessel walls. This offers the advantage of helping to prevent cardiovascular diseases.

Malic acid is effective in removing toxins from the liver, and because a healthy liver plays an important role in breaking down and metabolizing cholesterol, improved liver health consequently aids in managing blood cholesterol levels.

The enzyme food 100 includes 0.005 to 2 parts by weight of malic acid per 100 parts by weight of the enzyme food 100, and to maximize the efficacy derived from malic acid, it preferably includes 0.01 parts by weight of malic acid.

Chicory extract possesses functionalities including improving cholesterol levels, facilitating smooth bowel movements, and suppressing post-meal blood sugar increases. It refers to a product manufactured by extracting and refining the root of chicory or Cichorium intybus by using hot water. The extract must contain at least 90% dietary fiber.

Carotene abundantly contained in chicory extract exhibits excellent antioxidant activity by removing oxygen free radicals and inhibiting bodily cell aging. This offers the advantage of helping to prevent various lifestyle-related diseases.

Intybin contained in chicory extract provides chicory's characteristic bitter taste and helps aid digestion. Components in chicory, such as inulin and intybin, are effective for improving cholesterol levels and vascular health, thus offering the advantage of helping to prevent arteriosclerosis and hypertension.

Components such as intybin and beta-carotene in chicory extract are effective for anti-cancer activity. In addition, antioxidant components in chicory, including abundant polyphenols, exhibit inflammation-suppressing effects, offering the advantage of helping to treat skin inflammation.

Inulin, abundantly contained in chicory extract, is effective for controlling blood sugar levels, making it suitable for consumption by individuals with diabetes. Furthermore, abundant dietary fiber in chicory improves intestinal health and is effective for preventing constipation.

The root used for chicory extract has long been used medicinally for its tonic effect, proving effective as a restorative agent. Due to the detoxification action of chicory, chicory extract offers the advantage of being effective for improving liver function and treating icteric hepatitis.

In a method of manufacturing chicory extract, it is important to perform an extraction process in a way that maximizes the yield of key active components contained in chicory, such as inulin, polyphenols, intybin, beta-carotene, dietary fiber, and antioxidant components, without damage.

The method of manufacturing chicory extract includes an ingredient preparation operation involving washing and drying chicory roots and leaves, followed by grinding the dried chicory into powder, a hot-water extraction operation involving infusing the chicory powder in hot water at approximately 80 to 90° C. for a predetermined time period to extract water-soluble components such as inulin, an extract purification operation including a filtration process of removing unwanted impurities after the extraction and a concentration process of further concentrating the active ingredients, a concentrate drying operation involving producing powder from the concentrated extract via spray drying or freeze drying to enhance storage stability of the concentrated extract, and a quality testing operation involving checking the active component contents in the powdered chicory extract and assessing safety via microbial testing or the like.

During the chicory extract manufacturing process, it is preferable to add antioxidants or maintain low-temperature and light-shielded conditions to prevent oxidation of the extract. Furthermore, optimizing conditions such as extraction time, temperature, and solvent ratio is essential for maximizing the efficacy of the chicory extract.

The enzyme food 100 includes 0.005 to 2 parts by weight of the chicory extract per 100 parts by weight of the enzyme food 100, and to maximize the efficacy derived from the chicory extract, it preferably includes 0.01 parts by weight of the chicory extract.

Enzyme-treated stevia is an ingredient with improved sweetness quality, prepared by adding glucose to a stevia sweetener, and its sweetness profile is close to that of sugar, with low bitterness. It is stable to heat and acid, offering the advantage of minimal change in sweetness during processing or storage.

Enzyme-treated stevia is not readily utilized as a nutrient source for microorganisms and is substantially non-cariogenic in the oral cavity, thus not harming dental health. It also offers the advantage of not undergoing browning because it does not participate in the Maillard reaction (glycosylation reaction).

Being low in calories, enzyme-treated stevia helps lower the risk of obesity, lifestyle-related diseases, diabetes, hyperglycemia, and cavities. It also possesses antioxidant functions that help prevent aging and offers the advantage of being utilized in diets aimed at managing high blood pressure.

A method of manufacturing enzyme-treated stevia includes an ingredient preparation operation involving washing stevia leaves to remove impurities, an extraction operation involving soaking the washed leaves in water to extract desired sugar components (stevioside and rebaudioside), a filtration and concentration operation involving filtering the stevia extract to remove impurities and then preparing a stevia concentrate via vacuum concentration, an enzyme addition operation where enzymes such as glycosyl transferase and cyclodextrin glucanotransferase (CGTase) are added to improve taste and reduce bitterness, an enzyme reaction operation involving performing an enzyme reaction for 2 to 8 hours at 40 to 60° C., with strict control over temperature, hydrogen ion concentration index (pH), and reaction time, an enzyme removal operation involving removing residual enzymes via an inactivation process to enhance stability and safety, an impurity filtration operation involving removing impurities from the enzyme-treated stevia extract and concentrating it, a drying operation involving drying the concentrated extract into powder form by using a spray dryer or a freeze dryer, a powdering operation involving grinding the powdered stevia to produce a fine powder, and a quality testing operation involving inspecting the quality of the powdered enzyme-treated stevia.

Stevia, used for enzyme-treated stevia, is a natural sweetener extracted from stevia leaves, and is widely used as a sugar substitute for individuals requiring diabetes or weight management. Steviol glycosides, the main chemical components of stevia, exert various effects on health.

Unlike sugar, stevia does not raise blood sugar levels in the body. Steviol glycosides are not directly absorbed in the intestine but are mostly metabolized in the liver and do not affect glucose metabolism. Thus, stevia is effective for maintaining stable blood sugar levels.

Stevioside and rebaudioside, the main components of stevia, provide high sweetness but contain almost no calories, and because they are sweeter than sugar yet lower in calories, they are effective for weight management.

Steviol glycosides, the main chemical components of stevia, dilate blood vessels and promote diuretic action, and stevioside contributes to inhibiting the activity of angiotensin-converting enzyme (ACE). This offers the advantage of lowering blood pressure to stable levels in individuals with hypertension.

Antioxidant components contained in stevia, including flavonoids, neutralize oxygen free radicals, thereby reducing oxidative stress, and the anti-inflammatory effect of stevia inhibits the action of inflammation-causing substances, which helps prevent inflammatory diseases, improve cardiovascular health, prevent skin aging, and enhance immunity.

Unlike sugar, stevia does not promote the growth of oral bacteria, instead, it acts to inhibit the proliferation of harmful bacteria in the oral cavity. Therefore, it offers the advantage of being effective for preventing cavities and improving oral health.

Stevia regulates gastric acid secretion in the stomach and promotes the activity of digestive enzymes, thereby improving digestive function. It helps prevent indigestion and gastrointestinal disorders and improves the gut microbial environment, contributing to intestinal health.

When absorbed into the body, stevia offers the advantage of maintaining heart health by reducing levels of total cholesterol, triglycerides, and low-density cholesterol, while increasing high-density cholesterol.

The enzyme food 100 includes 0.005 to 2 parts by weight of enzyme-treated stevia per 100 parts by weight of the enzyme food 100, and to maximize the efficacy derived from enzyme-treated stevia, it preferably includes 0.01 parts by weight of enzyme-treated stevia.

The apple powder, fructo-oligosaccharides, psyllium husk powder, gluten-degrading lactic acid bacteria, and chicory extract, which are included in the enzyme food 100, are commonly rich in dietary fiber and prebiotics, and thus offer the advantages of aiding beneficial intestinal bacteria growth, improving intestinal health, and promoting digestion.

The gluten-degrading lactic acid bacteria, fructo-oligosaccharides, and apple powder, which are included in the enzyme food 100, increase beneficial intestinal bacteria and help activate immune cells. As intestinal health improves, the immune system strengthens, increasing resistance to external pathogens.

The psyllium husk, chicory, and guar gum, which are included in the enzyme food 100, help prevent overeating by slowing the digestion rate and maintaining satiety, and also contribute to blood sugar control by preventing rapid blood sugar spikes.

The apple powder, licorice concentrate powder, and vinegar powder, which are contained in the enzyme food 100, include antioxidant components, which help remove oxygen free radicals from the body, and polyphenol components contained in apples provide a strong antioxidant effect, contributing to reduced cell damage.

The isolated soy protein and amino acids, which are contained in the enzyme food 100, supplement the body's protein and aid recovery from muscle damage. They are effective for maintaining physical energy and reducing fatigue.

The apple powder, guar gum, psyllium husk powder, and vinegar powder, which are contained in the enzyme food 100, help lower blood cholesterol levels, and pectin contained in the apple powder, and the guar gum play roles in lowering cholesterol, while the vinegar powder contributes to blood pressure management.

In particular, the apple powder, dextrin, fructo-oligosaccharides, isolated soy protein, licorice concentrate powder, guar gum, vinegar powder, salt, psyllium husk powder, malic acid, chicory extract, and enzyme-treated stevia, which are contained in the enzyme food 100, are commonly specialized in normalizing cholesterol levels, and via this cholesterol level normalization, they offer the advantage of preventing heart diseases including arteriosclerosis, angina, and myocardial infarction, cerebrovascular diseases including stroke and hypertension, and metabolic syndrome.

### Embodiment 1

TABLE 2

| | Embodi- | Comparative | Comparative | Comparative |
|---|---|---|---|---|
| Ingredient | ment 1 | Example 1 | Example 2 | Example 3 |
| Enzyme | 38.8 | 42.5983 | 41.4050 | 42.7270 |
| preparation | | | | |
| Powder | 35.22 | 38.66 | 37.5840 | 38.7842 |
| preparation | | | | |
| Apple powder | 11.4 | 2.744 | 12.1651 | 2.7530 |
| Dextrin | 7.2 | 7.903 | 7.6832 | 7.9286 |
| Fructo- | 3.1 | 3.402 | 3.3080 | 3.4137 |
| oligosaccharides | | | | |
| Isolated soy | 2.1 | 2.305 | 2.2409 | 2.3125 |
| protein | | | | |
| Synthetic | 0.75 | 0.823 | 0.8003 | 0.8259 |
| flavoring agent | | | | |
| Amino acid | 0.4 | 0.439 | 5.9758 | 0.4404 |
| mixture powder | | | | |
| Licorice | 0.3 | 0.329 | 0.0106 | 0.0110 |
| concentrate | | | | |
| powder | | | | |
| Gluten-degrading | 0.2 | 0.219 | 0.2134 | 0.2202 |
| lactic acid | | | | |
| bacteria | | | | |
| Guar gum | 0.1 | 0.109 | 0.1067 | 0.1101 |
| Vinegar powder | 0.1 | 0.109 | 0.1067 | 0.1101 |
| Lactic acid | 0.1 | 0.109 | 0.1067 | 0.1101 |
| bacteria mixture | | | | |
| powder | | | | |
| Salt | 0.1 | 0.109 | 0.1067 | 0.1101 |
| Psyllium husk | 0.1 | 0.109 | 0.1067 | 0.1101 |
| powder | | | | |
| Malic acid | 0.01 | 0.0109 | 0.0106 | 0.011 |
| Chicory extract | 0.01 | 0.0109 | 0.0106 | 0.011 |
| Enzyme-treated | 0.01 | 0.0109 | 0.0106 | 0.011 |
| stevia | | | | |
| Total | 100 | 100 | 100 | 100 |

TABLE 3

| Score | Evaluation criteria |
|---|---|
| 5 | Very good |
| 4 | Good |
| 3 | Average |
| 2 | Poor |
| 1 | Very poor |

TABLE 4

| | | Comparative | Comparative | Comparative |
|---|---|---|---|---|
| | Embodiment 1 | Example 1 | Example 2 | Example 3 |
| Flavor | 4.7 | 4.2 | 3.8 | 3.2 |
| Aroma | 4.9 | 3.9 | 4.1 | 3.7 |
| Color | 4.8 | 4.6 | 4.5 | 4.2 |
| Texture | 4.5 | 4.2 | 4.8 | 4.6 |
| Overall | 4.5 | 4.1 | 4.0 | 3.4 |
| liking | | | | |

Referring to the table above, the enzyme food 100 manufactured according to Embodiment 1 includes 38.8 parts by weight of an enzyme preparation, 35.22 parts by weight of a powder preparation, 11.4 parts by weight of apple powder, 7.2 parts by weight of dextrin, 3.1 parts by weight of fructo-oligosaccharides, 2.1 parts by weight of isolated soy protein, 0.75 parts by weight of a synthetic flavoring agent, 0.4 parts by weight of amino acid mixture powder, 0.3 parts by weight of licorice concentrate powder, 0.2 parts by weight of gluten-degrading lactic acid bacteria, 0.1 parts by weight of guar gum, 0.1 parts by weight of vinegar powder, 0.1 parts by weight of lactic acid bacteria mixture powder, 0.1 parts by weight of salt, 0.1 parts by weight of psyllium husk powder, 0.01 parts by weight of malic acid, 0.01 parts by weight of chicory extract, and 0.01 parts by weight of enzyme-treated stevia.

The enzyme food 100 is manufactured in powder form by mixing 38.8 parts by weight of an enzyme preparation, 35.22 parts by weight of a powder preparation, 11.4 parts by weight of apple powder, 7.2 parts by weight of dextrin, 3.1 parts by weight of fructo-oligosaccharides, 2.1 parts by weight of isolated soy protein, 0.75 parts by weight of a synthetic flavoring agent, 0.4 parts by weight of amino acid mixture powder, 0.3 parts by weight of licorice concentrate powder, 0.2 parts by weight of gluten-degrading lactic acid bacteria, 0.1 parts by weight of guar gum, 0.1 parts by weight of vinegar powder, 0.1 parts by weight of lactic acid bacteria mixture powder, 0.1 parts by weight of salt, 0.1 parts by weight of psyllium husk powder, 0.01 parts by weight of malic acid, 0.01 parts by weight of chicory extract, and 0.01 parts by weight of enzyme-treated stevia.

Referring to Tables 3 and 4, a sensory evaluation of the enzyme food 100 manufactured according to Embodiment 1 was conducted with a panel of 30 participants, including 10 males and 20 females, and the participants' ages ranged from their 30s to their 60s, with an average age of 36.13 years.

The sensory evaluation scores for the enzyme food 100 manufactured according to Embodiment 1 corresponding to flavor, aroma, color, texture, and overall liking were 4.7, 4.9, 4.8, 4.5, and 4.5, respectively. It may be confirmed that the enzyme food 100 exhibited excellent overall flavor, aroma, color, texture, and overall liking.

### Comparative Example 1

Referring to the table above, the enzyme food manufactured according to Comparative Example 1 includes 42.5983 parts by weight of an enzyme preparation, 38.66 parts by weight of a powder preparation, 2.744 parts by weight of apple powder, 7.903 parts by weight of dextrin, 3.402 parts by weight of fructo-oligosaccharides, 2.305 parts by weight of isolated soy protein, 0.823 parts by weight of a synthetic flavoring agent, 0.439 parts by weight of amino acid mixture powder, 0.329 parts by weight of licorice concentrate powder, 0.219 parts by weight of gluten-degrading lactic acid bacteria, 0.109 parts by weight of guar gum, 0.109 parts by weight of vinegar powder, 0.109 parts by weight of lactic acid bacteria mixture powder, 0.109 parts by weight of salt, 0.109 parts by weight of psyllium husk powder, 0.0109 parts by weight of malic acid, 0.0109 parts by weight of chicory extract, and 0.0109 parts by weight of enzyme-treated stevia.

The enzyme food is manufactured in powder form by mixing 42.5983 parts by weight of an enzyme preparation, 38.66 parts by weight of a powder preparation, 2.744 parts by weight of apple powder, 7.903 parts by weight of dextrin, 3.402 parts by weight of fructo-oligosaccharides, 2.305 parts by weight of isolated soy protein, 0.823 parts by weight of a synthetic flavoring agent, 0.439 parts by weight of amino acid mixture powder, 0.329 parts by weight of licorice concentrate powder, 0.219 parts by weight of gluten-degrading lactic acid bacteria, 0.109 parts by weight of guar gum, 0.109 parts by weight of vinegar powder, 0.109 parts by weight of lactic acid bacteria mixture powder, 0.109 parts by weight of salt, 0.109 parts by weight of psyllium husk powder, 0.0109 parts by weight of malic acid, 0.0109 parts by weight of chicory extract, and 0.0109 parts by weight of enzyme-treated stevia.

Referring to Tables 3 and 4, a sensory evaluation of the enzyme food manufactured according to Comparative Example 1 was conducted with a panel of 30 participants, including 10 males and 20 females, and the participants' ages ranged from their 30s to their 60s, with an average age of 36.13 years.

The sensory evaluation scores for the enzyme food manufactured according to Comparative Example 1 corresponding to flavor, aroma, color, texture, and overall liking were 4.2, 3.9, 4.6, 4.2, and 4.1, respectively. It may be confirmed that the sensory evaluation scores for flavor, aroma, and overall liking of the enzyme food manufactured according to Comparative Example 1 were relatively significantly lower than those for the enzyme food 100 manufactured according to Embodiment 1.

Referring to Tables 2 to 4, it may be confirmed that the parts by weight of the apple powder included in Comparative Example 1 are relatively lower than the parts by weight of the apple powder included in Embodiment 1. Furthermore, it may be confirmed that the lower sensory evaluation scores for flavor, aroma, and overall liking in Comparative Example 1 are attributable to this lower amount of the apple powder.

### Comparative Example 2

Referring to the table above, the enzyme food manufactured according to Comparative Example 2 includes 41.4050 parts by weight of an enzyme preparation, 37.5840 parts by weight of a powder preparation, 12.1651 parts by weight of apple powder, 7.6832 parts by weight of dextrin, 3.3080 parts by weight of fructo-oligosaccharides, 2.2409 parts by weight of isolated soy protein, 0.8003 parts by weight of a synthetic flavoring agent, 5.9758 parts by weight of amino acid mixture powder, 0.0106 parts by weight of licorice concentrate powder, 0.2134 parts by weight of gluten-degrading lactic acid bacteria, 0.1067 parts by weight of guar gum, 0.1067 parts by weight of vinegar powder, 0.1067 parts by weight of lactic acid bacteria mixture powder, 0.1067 parts by weight of salt, 0.1067 parts by weight of psyllium husk powder, 0.0106 parts by weight of malic acid, 0.0106 parts by weight of chicory extract, and 0.0106 parts by weight of enzyme-treated stevia.

The enzyme food is manufactured in powder form by mixing 41.4050 parts by weight of an enzyme preparation, 37.5840 parts by weight of a powder preparation, 12.1651 parts by weight of apple powder, 7.6832 parts by weight of dextrin, 3.3080 parts by weight of fructo-oligosaccharides, 2.2409 parts by weight of isolated soy protein, 0.8003 parts by weight of a synthetic flavoring agent, 5.9758 parts by weight of amino acid mixture powder, 0.0106 parts by weight of licorice concentrate powder, 0.2134 parts by weight of gluten-degrading lactic acid bacteria, 0.1067 parts by weight of guar gum, 0.1067 parts by weight of vinegar powder, 0.1067 parts by weight of lactic acid bacteria mixture powder, 0.1067 parts by weight of salt, 0.1067 parts by weight of psyllium husk powder, 0.0106 parts by weight of malic acid, 0.0106 parts by weight of chicory extract, and 0.0106 parts by weight of enzyme-treated stevia.

Referring to Tables 3 and 4, a sensory evaluation of the enzyme food manufactured according to Comparative Example 2 was conducted with a panel of 30 participants, including 10 males and 20 females, and the participants' ages ranged from their 30s to their 60s, with an average age of 36.13 years.

The sensory evaluation scores for the enzyme food manufactured according to Comparative Example 2 corresponding to flavor, aroma, color, texture, and overall liking were 3.8, 4.1, 4.5, 4.8, and 4.0, respectively. It may be confirmed that the sensory evaluation scores for flavor, aroma, and overall liking of the enzyme food manufactured according to Comparative Example 2 were relatively significantly lower than those for the enzyme food 100 manufactured according to Embodiment 1.

Referring to Tables 2 to 4, it may be confirmed that the parts by weight of the licorice concentrate powder included in Comparative Example 2 are relatively lower than the parts by weight of the licorice concentrate powder included in Embodiment 1. Furthermore, it may be confirmed that the lower sensory evaluation scores for flavor, aroma, and overall liking in Comparative Example 2 are attributable to this lower amount of the licorice concentrate powder.

### Comparative Example 3

Referring to the table above, the enzyme food manufactured according to Comparative Example 3 includes 42.7270 parts by weight of an enzyme preparation, 38.7842 parts by weight of a powder preparation, 2.7530 parts by weight of apple powder, 7.9286 parts by weight of dextrin, 3.4137 parts by weight of fructo-oligosaccharides, 2.3125 parts by weight of isolated soy protein, 0.8259 parts by weight of a synthetic flavoring agent, 0.4404 parts by weight of amino acid mixture powder, 0.0110 parts by weight of licorice concentrate powder, 0.2202 parts by weight of gluten-degrading lactic acid bacteria, 0.1101 parts by weight of guar gum, 0.1101 parts by weight of vinegar powder, 0.1101 parts by weight of lactic acid bacteria mixture powder, 0.1101 parts by weight of salt, 0.1101 parts by weight of psyllium husk powder, 0.011 parts by weight of malic acid, 0.011 parts by weight of chicory extract, and 0.011 parts by weight of enzyme-treated stevia.

The enzyme food is manufactured in powder form by mixing 42.7270 parts by weight of an enzyme preparation, 38.7842 parts by weight of a powder preparation, 2.7530 parts by weight of apple powder, 7.9286 parts by weight of dextrin, 3.4137 parts by weight of fructo-oligosaccharides, 2.3125 parts by weight of isolated soy protein, 0.8259 parts by weight of a synthetic flavoring agent, 0.4404 parts by weight of amino acid mixture powder, 0.0110 parts by weight of licorice concentrate powder, 0.2202 parts by weight of gluten-degrading lactic acid bacteria, 0.1101 parts by weight of guar gum, 0.1101 parts by weight of vinegar powder, 0.1101 parts by weight of lactic acid bacteria mixture powder, 0.1101 parts by weight of salt, 0.1101 parts by weight of psyllium husk powder, 0.011 parts by weight of malic acid, 0.011 parts by weight of chicory extract, and 0.011 parts by weight of enzyme-treated stevia.

Referring to Tables 3 and 4, a sensory evaluation of the enzyme food manufactured according to Comparative Example 3 was conducted with a panel of 30 participants, including 10 males and 20 females, and the participants' ages ranged from their 30s to their 60s, with an average age of 36.13 years.

The sensory evaluation scores for the enzyme food manufactured according to Comparative Example 3 corresponding to flavor, aroma, color, texture, and overall liking were 3.2, 3.7, 4.2, 4.6, and 3.4, respectively. It may be confirmed that the sensory evaluation scores for flavor, aroma, and overall liking of the enzyme food manufactured according to Comparative Example 3 were relatively significantly lower than those for the enzyme food 100 manufactured according to Embodiment 1.

Referring to Tables 2 to 4, it may be confirmed that the respective parts by weight of the apple powder and the licorice concentrate powder included in the enzyme food of Comparative Example 3 are relatively lower than those in the enzyme food 100 of Embodiment 1. Furthermore, it may be confirmed that the lower sensory evaluation scores for flavor, aroma, and overall liking in Comparative Example 3 are attributable to these lower amounts of the apple powder and the licorice concentrate powder.

Although preferred embodiments of the present disclosure have been described above with reference to the accompanying drawings, the embodiments described herein and the configurations illustrated in the drawings are merely examples of preferred embodiments of the present disclosure and do not represent the entire technical spirit of the present disclosure. Therefore, it should be understood that various equivalents and modifications that may replace these elements could exist at the time of filing the present application. Therefore, the embodiments described above are to be considered in all respects as illustrative and not restrictive. The scope of the present disclosure is indicated by the appended claims rather than by the foregoing description. All changes or modifications that come within the meaning and range of equivalency of the claims are therefore intended to be embraced within the scope of the present disclosure.

### PRIOR ART DOCUMENTS

### Patent Documents

(Patent Document 1) Korean Patent Registration No. 10-1866468
(Patent Document 2) Korean Patent Publication No. 10-2325467

### LIST OF REFERENCE NUMERALS FOR MAJOR ELEMENTS

100: Enzyme food

1. An enzyme food comprising:
38.8 parts by weight of an enzyme preparation including teff enzyme, 35.22 parts by weight of a powder preparation, 11.4 parts by weight of apple powder, 7.2 parts by weight of dextrin, 3.1 parts by weight of fructo-oligosaccharides, 2.1 parts by weight of isolated soy protein, 0.75 parts by weight of a synthetic flavoring agent, 0.4 parts by weight of amino acid mixture powder, 0.3 parts by weight of licorice concentrate powder, 0.2 parts by weight of gluten-degrading lactic acid bacteria, 0.1 parts by weight of guar gum, 0.1 parts by weight of vinegar powder, 0.1 parts by weight of lactic acid bacteria mixture powder, 0.1 parts by weight of salt, 0.1 parts by weight of psyllium husk powder, 0.01 parts by weight of malic acid, 0.01 parts by weight of chicory extract, and 0.01 parts by weight of stevia,
wherein the powder preparation is indigestible maltodextrin, Bacillus subtilis, Aspergillus oryzae, cereal mixture powder, or germinated brown rice.

2-6. (canceled)

7. The enzyme food of claim 1, wherein the stevia is enzyme-treated stevia manufactured by adding glycosyl enzyme to stevia extract and fermenting a resulting mixture.

8. The enzyme food of claim 7, wherein the lactic acid bacteria mixture powder comprises Lactobacillus plantarum, Lactobacillus rhamnosus, Lactobacillus casei, Lactobacillus acidophilus, Lactobacillus paracasei, Lactobacillus salivarius, and Lactobacillus gasseri.

9. (canceled)
